(19) United States
(12) Patent Application Publication
(10) Pub. No.: US 2018/0195081 A1
(43) Pub. Date: Jul. 12, 2018

(54) TRANSGENIC PLANTS EXPRESSING LEAF-SPECIFIC PROTEINS

(71) Applicant: University of Hawaii, Honolulu, HI (US)

(72) Inventors: Michael Shintaku, Honolulu, HI (US); Lukas Kambic, Honolulu, HI (US)

(21) Appl. No.: 15/730,225

(22) Filed: Oct. 11, 2017

Related U.S. Application Data
(60) Provisional application No. 62/406,901, filed on Oct. 11, 2016.
(60) Provisional application No. 62/407,458, filed on Oct. 12, 2016.

(51) Int. Cl. C12N 15/82; C07K 14/43

(52) U.S. Cl. CPC C12N 15/8251; C07K 14/43; C12N 15/8225; C12N 15/8205; C12N 15/8207

(57) ABSTRACT

The invention relates to transgenic plants expressing the leaf-specific proteins.

### CROSS REFERENCE TO RELATED APPLICATIONS

This application claims the benefit of U.S. Provisional Application No. 62/406,901, filed on Oct. 11, 2016, and U.S. Provisional Application No. 62/407,458, filed on Oct. 12, 2016, both entitled Plants Expressing the Miraculin Protein. The entire contents of the foregoing are hereby incorporated by reference.

### FIELD OF THE INVENTION

The invention disclosed herein generally relates to transgenic plants expressing the leaf-specific proteins including but not limited to the miraculin protein or homologs of the miraculin protein, and methods for producing high-quality and/or large quantities of leaf-specific proteins including but not limited to the miraculin protein or homologs of the miraculin protein.

### BACKGROUND

Miraculin is a flavor-altering protein extracted from the fruit of a miracle fruit plant (Synsepalum dulcificum).

Miraculin has an effect of altering sour and bitter flavors of a food to a sweet flavor. For example, for about 30 minutes after eating a miracle berry, sour foods such as lemons, limes and vinegar will taste sweet to a person. As another example, tabasco will taste hot and sweet. Sweetness of 0.1 μM miraculin induced by 0.1M of citric acid was equal to 400,000 times that of sucrose. The sweet taste continues for several hours after consumption of 0.1 mg miraculin (the amount in 0.5-2 g of berries).

Because of such effects, miraculin can be used in medical and food applications. For example, since miraculin can deliver a very sweet flavor with very few calories, the protein can be used a supplement for diabetics and/or weight loss.

However, production of miraculin is limiting. It takes 3-4 years from planting for a miracle berry plant, to naturally to produce berries. Furthermore, berry production is limited to year-round tropical conditions. In addition, the yield is variable and sporadic and the fruits are highly perishable. Generally, 50-200 mg miraculin can be obtained from one kilogram of berries.

The invention disclosed herein relates to transgenic plants expressing the miraculin protein and methods for producing the miraculin protein. Other embodiments of the invention relate to expressing other proteins having limited native availability or extractability, high potential value/demand, and good suitability for overexpression in leaves of transgenic plants.

### SUMMARY OF THE INVENTION

Embodiments of the invention relate to a plant including a protein expressed in leaves thereof. The protein can be a heterologous miraculin protein, or a fragment, analog, or variant of a miraculin protein

In some embodiments, the plant can include an integrated gene construct comprising a sequence encoding the miraculin protein. In some embodiments, the sequence encoding the miraculin protein can be identical to, a fragment of, related to, or derived from, the miraculin gene of Synsepalum dulcificum.

In some embodiments, the construct of the plant can further include a promoter sequence heterologous to both the host plant and to the miraculin. In some embodiments, the construct can further include a transcriptional termination sequence heterologous to the promoter, the miraculin, and the host plant. In some embodiments, the promotor can include an operable portion of a plastocyanin promoter region from Pisum sativum. In some embodiments, the terminator can include an operable portion of a terminator/polyadenylation signal from the heat shock protein 18.2 gene of Arabidopsis thaliana.

In some embodiments, the plant can be lettuce, arugula, beets, bok choy, brussels sprouts, cabbage, chard, collards, dandelion, kale, spinach, watercress, or the like.

Some embodiments of the invention relate to a method of expressing a protein in a leaf of a heterologous plant. The method can include the steps of: providing a gene construct including a sequence encoding the protein under control of a leaf-specific promoter; genetically transforming cells of a plant with the construct; regenerating differentiated plants from the transformed cells, the differentiated plants having leaves. The protein can be expressed in the leaves of the regenerated plants with this method.

For example, some embodiments of the invention relate to a method of expressing miraculin or a homolog of miraculin in a leaf of a heterologous plant. The method can include the steps of: providing a gene construct comprising a sequence encoding a miraculin protein or a fragment, analog, or variant thereof under control of a leaf-specific promoter; genetically transforming cells of a plant with the construct; regenerating differentiated plants from the transformed cells, the differentiated plants having leaves. Miraculin or a homolog of miraculin can be expressed in the leaves of the regenerated plants with this method. In some embodiments, the plant can be lettuce, arugula, beets, bok choy, brussels sprouts, cabbage, chard, collards, dandelion, kale, spinach, watercress, or the like.

In some embodiments, the plant transformation can be mediated by biolistic delivery of the construct to the cells. In some embodiments, the construct can include a targeting sequence sufficient to direct targeting of the protein to a subcellular compartment.

In some embodiments, the method further includes screening and selecting plants regenerated plants on the basis of a property of the miraculin. In some embodiments the property is selected from the group consisting of protein abundance, relative sweetness effect, relative masking effect, relative taste-altering effect and persistence of effect.

### BRIEF DESCRIPTION OF THE DRAWINGS

FIG. 1 depicts an example of a cassette used for leaf expression of miraculin.

### DETAILED DESCRIPTION OF THE INVENTION

Unless otherwise noted, terms are to be understood according to conventional usage by those of ordinary skill in the relevant art.

Embodiments of the invention relate to transgenic plants expressing miraculin or a homolog of miraculin, or another protein suitable for expression and/or accumulation in edible leaves. The plants can be, for example lettuce, arugula, beets, bok choy, brussels sprouts, cabbage, chard, collards, dandelion, kale, spinach, watercress, Claytonia (miner's lettuce), sorrel, purslane and the like.

The transgene construct contains a promotor region. In some embodiments, the construct can include a truncated plastocyanin promoter region from Pisum sativum. This promoter provides various desirable properties for the construct. First, it is known to be expressed specifically in the leaves and other green parts of dicot plants and therefore provides the desired ability for the miraculin protein to be very simply grown, harvested, and used in edible plant leaves. Second, this promoter permits overexpression of the miraculin gene and therefore permits abundant accumulation of the miraculin protein in an easily harvestable form for downstream uses. Third, the promoter originates from a familiar and non-allergenic edible plant, which can help to alleviate anxiety in people who are unfamiliar with or wary of plant biotechnology. Fourth, the leaf-specific expression pattern eliminates “wasted” protein production in non-edible or non-harvestable plant parts, such as roots and stems. Fifth, the promoter does not contain sequence elements from plant pests, so its use in transgenic plants does not constitute a trigger for regulation by USDA/APHIS, providing a significant advantage in terms of lower regulatory costs. Sixth, in combination with the noted terminator sequence, the promoter has demonstrated the ability to maintain stable expression over at least 4 seed generations in Lactuca sativa. Likewise, other leaf-specific promoters can be used in making analogous constructs that are within the scope of some embodiments of the invention. They include, for example, the alfalfa RAc promoter (Potenza et al. 2004. Targeting transgene expression in research, agricultural and environmental applications: Promoters used in plant transformation. In Vitro Cellular and Developmental Biology-Plant 40:1-22), the pea rbcS-3A promoter (Gilmartin P. M. and Chua, N. H. 1990. Spacing between GT-1 binding sites within a light-responsiver element is critical for transcriptional activity. Plant Cell 2:447-455), the Arabidopsis CAB2 promoter (Carre, I. A. and Kay, S. A. 1995. Multiple DNA-protein complexes at a circadian-regulated promoter element. Plant Cell 7:2039-2051). Each of the forgoing citations is incorporated by reference in its entirety.

The transgene construct can contain a terminator signal. In some embodiments, the construct can include a truncated terminator/polyadenylation signal from the heat shock protein 18.2 gene of Arabidopsis thaliana. This terminator sequence has the advantage of small size, origin in an edible plant, and an absence of plant pest components. In some embodiments, the construct can employ a native terminator sequence from miraculin or a homolog thereof. (Nagaya et al. 2009. The HSP terminator of A. thaliana increases gene expression in plant cells. Plant and Cell Physiology 51:328-332). The forgoing citation is incorporated by reference in its entirety.

Testing for transformation is conventional, typically with a selectable marker such as, for example, NPTII, conferring antibiotic resistance upon transformed cells. Alternatives to selectable markers for efficient confirmation of transformation include screening with polymerase chain reaction and organoleptic bioassay. In some embodiments, an NPTII selectable marker gene can be transcriptionally regulated by a ubiquitin-3 promoter and terminator from Solanum tuberosum. Any plant selectable marker system can be employed, including but not limited to hygromycin resistance, altered saccharide metabolism, and fluorescent marker proteins. Marker cassettes can be co-transformed and need not be linked to the miraculin expression cassette. Other selectable markers include: hygromycin phosphotransferase, gentamycin aminotransferase, streptomycin phosphotransferase and phosphinothricin acetyltransferase (Ziemienowicz, A. 2001. Plant Selectable Markers and Reporter Genes, Acta Physiologiae Plantarum 23:363-374). The forgoing citation is incorporated by reference in its entirety.

The core of the transgene construct of the miraculin coding sequence. This sequence was obtained from a miracle berry (Synsepalum dulcificum) plant and is known in the art (Genbank Accession AB512278). (Masuda et al. 1995. Cloning and sequencing of a cDNA encoding a taste-modifying protein, miraculin. Gene. 161:175-177). The forgoing citation is incorporated by reference in its entirety.

The transgenic plant can produce high levels of functional miraculin or homologs. The miraculin can be in the leaves and/or greens of the plant. Miraculin in lettuce leaves can be produced in quantities comparable to that found in miracle berry fruit. In some embodiments, miraculin modified to increase or reduce its binding affinities, vulnerability to proteases, or its thermal stability can be expressed. Plant tissue concentrations in the range of about 0.01 to 1 mg/g of recombinant miraculin or homologs can be produced by the expression system described, wherein mg/g indicates mg miraculin per gram dry mass of leaf tissue.

The transgenic plant can contain one or more copies of a transgene construct. The construct can include a gene encoding a protein with flavor-altering properties. In some embodiments, the construct can include part of or the full miraculin coding region from Synsepalum dulcificum (SEQ ID NO. 1). In some embodiments, the construct can have 50%, 60%, 70%, 80%, 90% or 100% sequence identity to the full miraculin coding region. The construct can include the 3′ signal peptide region for the miraculin coding region, and/or the 5′ untranslated region of the miraculin gene or homologs thereof.

In some embodiments, the gene can encode a protein that includes full or part of the amino acid sequence of miraculin (SEQ ID NO. 2). For example, the gene can encode an amino acid sequence having 50%, 60%, 70%, 80%, 90% or 100% identity with the full amino acid sequence of miraculin.

Homologs of miraculin are defined as proteins similar to miraculin in sequence and have flavor-altering properties, including naturally occurring genes as well as intentionally altered functional variants. For example, homologs occur in pea and tomato, but have debatable activity. In this disclosure, in a context in which such usage is logical, the terms “miraculin” and “miraculin homolog” are interchangeable.

In some embodiments, the gene encoding a protein with flavor-altering properties can encode an amino acid sequence derived from full or part of the amino acid sequence of miraculin by deletion, substitution, or addition of one or more amino acids.

Expression of miraculin in the transgenic plants can be stable. Expression can last over multiple generations. Expression can be determined by bioassay of leaves, or with an antibody-based assay (Hirai et al. 2009. Miraculin, a taste-modifying protein is secreted into intercellular spaces in plant cells. Journal of Plant Physiology 167:209-215). In some embodiments, the miraculin coding region can be modified, for example, to direct the targeting and accumulation of the protein to a subcellular compartment or to the cytosol. (Pereira et al. 2014. Delivering of proteins to the plant vacuole—an update. Int J Mol Sci 15(5):7611-7623. Each of the forgoing citations is incorporated by reference in its entirety.

Use of some embodiments of the invention can be as simple as eating the leaves expressing the miraculin. Additionally, leaves can be processed by use of extraction, juicing, concentration, fractionation, freeze-drying, purification, lyophilization, pasteurization, or any combination thereof, and the like.

Extracted miraculin can be used in medical and food applications. For example, the protein can be used as a supplement for diabetics and/or weight loss. As another example, the protein can relieve the unpleasant metallic taste associated with chemotherapy (Wilken et al. 2012 Pilot study of “miracle fruit” to improve food palatability for patients receiving chemotherapy. Clinical Journal of Oncology Nursing 16:E173-E177). The foregoing citation is incorporated by reference in its entirety.

Leaves and/or leaf components can be used in other products or preparations including but not limited to: edible preparations such as powders, juices, drops, flavor shots, smoothies, salads, sandwiches, or the like; topical preparations such as creams, gels, poultices, patches, plasters, or the like; and formulated to be taken or delivered internally such as into capsules, pills, or other internal routes. (WO 2016103163 A1; WO 2015177522 A1). Each of the forgoing citations is incorporated by reference in its entirety.

Some embodiments of the invention relate a method for producing the transgenic plant expressing miraculin or a homologue of miraculin. In some embodiments, the method can include introducing the transgene construct into a plant cell. Genetic transformation of the plant or plants, within the scope of embodiments of the invention, can be by conventional means including, for example, plasmid transformation via Agrobacterium tumefaciens, biolistics transformation, electroporation, microinjection, and the like. Insertion of the construct or desired portions thereof into via targeted genome editing can be accomplished by, for example, CRISPR/Cas9 or TALEN approaches, or the like. (Gelvin, S. B. 2003. Agrobacterium-mediated plant transformation: the biology behind the “gene-jockeying” tool. Microbiol Mol Biol Rev. 67-1. Neuhaus, G., G. Spangenberg 1990. Plant transformation by microinjection techniques. Physiologia Plantarum, 79:213-217. Yao et al., 2006. Low copy number gene transfer and stable expression in a commercial wheat cultivar via particle bombardment. Journal of Experimental Botany, 57:3737-3746. Bates, G. W. 1999. Plant transformation via protoplast electroporation. In: Plant Cell Culture Protocols. Methods in Molecular Biology, 111:359-366). Each of the forgoing citations is incorporated by reference in its entirety.

In some embodiments, plant transformation can be performed via cotyledon inoculation. In some embodiments, recombinant DNA can be introduced into plant cells via particle bombardment methods, including the use of minimal cassettes or plasmids with or without selectable marker cassettes. In some embodiments, inoculation can include use of Agrobacterium strain LBA4404 harboring the vector pBINPLUS/ARS.

Mature leaves, shoots or cotyledons of lettuce (Lactuca sativa) can be bombarded with DNA construct(s) attached to gold or tungsten particles. Alternatively, lettuce cell cultures can be agitated with silicon carbide whiskers in a solution containing DNA construct(s). After 72 hours of recovery in vitro on Murashige and Skoog (MS) media containing 0.1 mg/L naphthaleneacetic acid and 0.1 mg/L benzylaminopurine, explants or cells can be transferred to regeneration/selection media containing 0.1 mg/L naphthaleneacetic acid, 0.1 mg/L benzylaminopurine, and 50 mg/L kanamycin sulfate or other selection agent, resulting in induction of transgenic callus growth. Resulting callus tissue can be transferred to fresh media every ten days until fertile shoots regenerate. Resulting shoots can be excised and transferred to MS media without growth regulators to allow root development prior to acclimatization to ambient ex-vitro conditions to permit evaluation and seed production. Alternatively, in some embodiments, mature leaves, shoots, or cotyledons of Lactuca sativa can be inoculated with Agrobacterium tumefaciens harboring suitable T-plasmids that carry the described DNA construct(s) within their T-DNA borders, followed by 72 hours of co-cultivation on MS media containing 0.1 mg/L naphthaleneacetic acid and 0.1 mg/L benzylaminopurine and transfer to selection/regeneration media containing 0.1 mg/L naphthaleneacetic acid, 0.1 mg/L benzylaminopurine, 150 mg/L Timentin and 50 mg/L kanamycin sulfate or other selection agent. Resulting shoots can be excised and transferred to MS media without growth regulators to allow root development prior to acclimatization to ambient ex-vitro conditions to permit evaluation and seed production. (Takeuchi et al. 1992. Plant transformation: a simple particle bombardment device based on flowing helium. Plant Molecular Biology, 18:835-839. Dandakar, A. M. and H. J. Fisk 2004. Plant Transformation: Agrobacterium-Mediated Gene Transfer. In: Methods in Molecular Biology vol 286 Transgenic Plants: Methods and Protocols:35-46). Each of the forgoing citations is incorporated by reference in its entirety.

In some embodiments, fertile shoots can be regenerated from calli in vitro. Regenerating the transgenic lettuce employing conventional approaches and/or adapting analogous approaches known in the art for other genera.

Selection of regenerated transformants is conventional, breeding of transformants can be conducted to eliminate selectable markers, minimize and select against undesirable somaclonal variants, and to transfer transgene(s) to desirable genetic backgrounds.

Determination of desirable transformants can be assayed by various approaches. In a simple approach for miraculin, a standard size leaf section from several transformants is removed and tasted, and the desirability of the taste or effect of the miraculin present in the leaf section is used as a qualitative proxy for a quantitative assessment of expression level of the protein. Evaluation can be determined by a taste panel or the like. (WO 2012054743 A2, US 20100029786 A1, WO 2016103163 A1). Each of the forgoing citations is incorporated by reference in its entirety.

In some embodiments, a form of expressed miraculin having a persistence of effect that is longer or shorter than normal can be beneficial and can be obtained by screening transformants for this characteristic. In some embodiments, the persistence effect can be of a duration of one minute or less, 5, 10, 15, 20, 25, 30, 35, 40, 45, 50, or 60 minutes or more.

The taste test can be correlated with actual expression levels via actual quantitative testing if desired. Such testing can take the form of protein immunoblots (western blots) or other molecule-specific quantitative or semi-quantitative assays as desired. Since in most cases this is only for screening among transformants and selecting the best ones for further study, breeding, and selection, rigorous quantitative analysis is generally not considered necessary. However, when/if such analysis is necessary, it is accessible by use of approaches that are within the level of knowledge and skill in the art.

For further specific discussion of qualitative, semi-quantitative, and quantitative analysis of miraculin and/or other expressed proteins, see the EXAMPLES section, below.

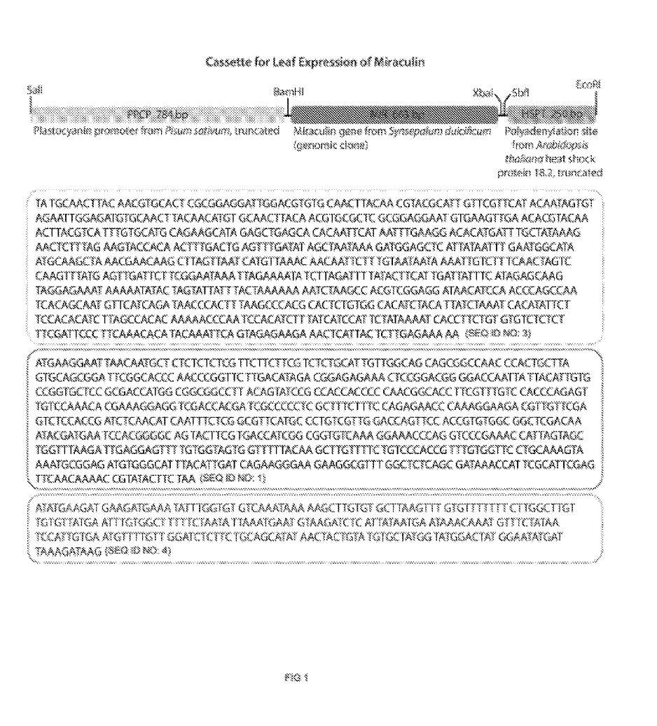

In other embodiments of the invention, the construct shown in FIG. 1 is modified to insert a sequence capable of encoding a different protein desirable for expression in plant leaves. While numerous proteins can be considered possible candidates for such a modification, the proteins having the highest value in this system are those that have some basic similarities to miraculin: they are relatively rare and/or hard to recover or extract from their native source and/or somewhat unstable in their native source; they are suitable for transgenic expression and overexpression (e.g., their overexpression does not cause harm to the plant cell or create extraction or recovery difficulties that cannot be solved); they are sufficiently stable in a plant leaf that they can accumulate in the leaf to useful levels; their value as transgenically expressed proteins creates an economic justification for making the transgenic plants, selecting the best expressors, growing progeny and so on. Non-limiting examples of such proteins are: brazzien, pentadin, monellin, thaumatin, and zika virus capsid protein.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

Lettuce cotyledons are inoculated with Agrobacterium tumefaciens containing the pBINLUS/ARS vector with a construct (construct 1) between its T-DNA borders consisting of a truncated plastocyanin promoter from Pisum sativum, a genomic clone of the transcribed region of the miraculin gene from Synsepalum dulcificum, and a truncated heat shock protein 18.2 terminator from Arabidopsis thaliana. Plants are regenerated from transformed cells as described in [0034]. Regenerated plants are screened with reverse transcriptase-PCR reaction to detect presence of miraculin mRNA. Leaves of plants with detectable miraculin mRNA are tested using organoleptic bioassay: leaves are chewed to allow plant fluids to contact the tongue, followed by tasting of a 0.02 M solution of citric acid to evaluate altered perception of sweetness. Transformation events demonstrating desired activity are grown for multiple generations to evaluate expression stability and transgene insertion site(s) are mapped.

### Example 2

Lettuce cotyledons are bombarded with gold or tungsten particles coated in nucleic acids including (construct 1) and a selectable marker cassette. Plants are regenerated from transformed cells as described in [0034]. Regenerated plants are screened with reverse transcriptase-PCR reaction to detect presence of miraculin mRNA. Leaves of plants with detectable miraculin mRNA are tested using organoleptic bioassay: leaves are chewed to allow plant fluids to contact the tongue, followed by tasting of a 0.02 M solution of citric acid to evaluate altered perception of sweetness. Transformation events demonstrating desired activity are grown for multiple generations to evaluate expression stability and transgene insertion site(s) are mapped. Selectable marker gene(s) are removed by identifying null segregant progeny using PCR. High-performing insertion events are introgressed into desired lettuce varieties.

### Example 3

Lettuce cotyledons are bombarded with gold or tungsten particles coated in nucleic acids, including (construct 1) as described above with the addition of 1000-bp homologous arms that hybridize stringently with a desired genomic insertion target site, Cas9 mRNA, gRNA(s) targeting the desired genomic insertion target site, and an unlinked minimal selectable marker cassette. Plants are regenerated from transformed cells as described in [0034]. Regenerated plants are screened with reverse transcriptase-PCR reaction to detect presence of miraculin mRNA. Leaves of plants with detectable miraculin mRNA are tested using organoleptic bioassay: leaves are chewed to allow plant fluids to contact the tongue, followed by tasting of a 0.02 M solution of citric acid to evaluate altered perception of sweetness. Transformed lines demonstrating desired activity are grown for multiple generations to evaluate expression stability and transgene insertion site(s) are mapped. Selectable marker gene(s) are removed by identifying null segregant progeny using PCR. High-performing insertion events are introgressed into desired lettuce varieties.

### Example 4

It is known that in typical plant transformation, there is some amount of normal variability of copy number of transgenes inserted, insertion positions, and the like, that can have a significant effect on expression of the transgene. Likewise, it is possible that in some plant transformant lines, the transformation event itself and/or a certain amount of somaclonal variation introduced by the regeneration process can result in other sources of variability in the phenotype of the transgenic plants. Such variability is typically addressed and analyzed in terms of overall expression/accumulation level of the protein encoded by the transgene. However, other assays for other kinds of phenotypic variability can also be useful.

A plant transformation protocol is conducted and numerous cells are transformed resulting in numerous individual calli, from which whole plants are regenerated. Each plant is given an individual tracking identifier and is, at an appropriate stage, subject to further selection such that all plants displaying unacceptable lack of vigor or morphological irregularities are eliminated. Plants appearing to be morphologically normal and vigorous in growth are maintained for further evaluation. As part of this evaluation, standardized portions of leaves of each transformant line are evaluated in a taste panel and ranked in terms of sweetness or other desired gustatory criteria and all plant transformant lines are ranked from highest to lowest in terms of this characteristic. To the extent that the highest ranking correlates with the highest level of expression or best overall properties of the expressed protein, phenotypically, within the leaves, this simple qualitative assay and ranking can be sufficient for determining which lines are preferable for further maintenance and propagation.

In addition, the same large number of initial transformant lines that were not eliminated in the initial vigor/morphology screen, can also be tested and ranked for other criteria considered to be commercially useful. For example, while the typical duration of the sweetness or masking effect of miraculin is understood to be approximately 30 minutes there can be commercial value in having an effect that has a shorter or longer duration of persistence, as compared to the normal duration. A taste panel can evaluate duration of the persistence of the sweetness or masking effect by ingesting a standard leaf sample followed by a timecourse of tasting a standard solution whose flavor is known to be masked by the effects of miraculin. Each panel participant tastes the solution at time points of, for example, 1 minute, 3 minutes, 6 minutes, and so on, until the masking effect is gone and the true taste of the standard solution is detected by the participant, and the time point is logged. With proper controls and randomization and sample size, such a taste panel and timecourse analysis permits ranking the transformant lines in terms of the duration of persistence of the miraculin effect, and lines showing unusually short duration or unusually long duration can be selected for further evaluation and breeding.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

MIRACULIN FROM SYNSEPALUM DULCIFICUM, WILD TYPE
TRANSCRIBED REGION
SEQ ID NO. 1
atgaaggaat taacaatgct ctctctctcg ttcttcttcg
tctctgcatt gttggcagca gcggccaacc cactgcttag
tgcagcggat tcggcaccca acccggttct tgacatagac
ggagagaaac tccggacggg gaccaattat tacattgtgc
cggtgctccg cgaccatggc ggcggcctta cagtatccgc
caccaccccc aacggcacct tcgtttgtcc acccagagtt
gtccaaacac gaaaggaggt cgaccacgat cgccccctcg
ctttctttcc agagaaccca aaggaagacg ttgttcgagt
ctccaccgat ctcaacatca atttctcggc gttcatgccc
tgtcgttgga ccagttccac cgtgtggcgg ctcgacaaat
acgatgaatc cacggggcag tacttcgtga ccatcggcgg
tgtcaaagga aacccaggtc ccgaaaccat tagtagctgg
tttaagattg aggagttttg tggtagtggt ttttacaagc
ttgttttctg tcccaccgtt tgtggttcct gcaaagtaaa
atgcggagat gtgggcattt acattgatca gaagggaaga
aggcgtttgg ctctcagcga taaaccattc gcattcgagt
tcaacaaaac cgtatacttc taa
SEQ ID NO. 2
MIRACULIN (WILD-TYPE) AMINO ACID SEQUENCE
MKELTMLSLSFFFVSALLAAAANPLLSAADSAPNPVLDIDGEKL
RTGTNYYIVPVLRDHGGGLTVSATTPNGTFVCPPRVVQTRKEVDHDRPLA
FFPENPKEDVVRVSTDLNINFSAFMPCRWTSSTVWRLDKYDESTGQYFVT
IGGVKGNPGPETISSWFKIEEFCGSGFYKLVFCPTVCGSCKVKCGDVGIY
IDQKGRRRLALSDKPFAFEFNKTVYF

What is claimed is:

1. A plant comprising a heterologous miraculin protein, or a fragment, analog, or variant of a miraculin protein, expressed in leaves thereof.

2. The plant of claim 1, wherein the plant comprises an integrated gene construct comprising a sequence encoding the miraculin protein.

3. The plant of claim 2, wherein the sequence encoding the miraculin protein is identical to, a fragment of, related to, or derived from, the miraculin gene of Synsepalum dulcificum.

4. The plant of claim 2, wherein the construct further comprises a promoter sequence heterologous to both the host plant and to the miraculin.

5. The plant of claim 4, wherein the construct further comprises a transcriptional termination sequence heterologous to the promoter, the miraculin, and the host plant.

6. The plant of claim 4, wherein the promoter comprises an operable portion of a plastocyanin promoter region from Pisum sativum.

7. The plant of claim 5, wherein the terminator comprises an operable portion of a terminator/polyadenylation signal from the heat shock protein 18.2 gene of Arabidopsis thaliana.

8. The plant of claim 1, wherein the plant is selected from the group consisting of: lettuce, arugula, beets, bok choy, brussels sprouts, cabbage, chard, collards, dandelion, kale, spinach, and watercress.

9. A method of expressing miraculin in a leaf of a heterologous plant comprising the steps of:
providing a gene construct comprising a sequence encoding a miraculin protein, or a fragment, analog, or variant thereof, under control of a leaf-specific promoter;
genetically transforming cells of a plant with the construct;
regenerating differentiated plants from the transformed cells, the differentiated plants having leaves;
wherein the miraculin is expressed in the leaves of the regenerated plants.

10. The method of claim 9, wherein the plant is selected from the group consisting of: lettuce, arugula, beets, bok choy, brussels sprouts, cabbage, chard, collards, dandelion, kale, spinach, and watercress.

11. The method of claim 9, wherein the plant transformation is mediated by biolistic delivery of the construct to the cells.

12. The method of claim 9, wherein the construct comprises a targeting sequence sufficient to direct targeting of the protein to a subcellular compartment.

13. The method of claim 9, further comprising screening and selecting plants regenerated plants on the basis of a property of the miraculin.

14. The method of claim 13, wherein the property is selected from the group consisting of protein abundance, relative sweetness effect, relative masking effect, relative taste-altering effect and persistence of effect.


SEQUENCE LISTING (4 sequences)

SEQ ID NO: 1 - DNA, 661 bp, Artificial Sequence
  Synsepalum dulcificum
atgaaggaattaacaatgctctctctctcgttcttcttcgtctctgcattgttggcagcagcggccaacccactgcttagtgcagcggattcggcacccaacccggttcttgacatagacggagagaaactrcggacggggaccaattattacattgtgccggtgctccgcgaccatggcggcggccttacagtatccgccaccacccccaacggcaccttcgtttgtccacccagagttgtccaaacacgagaggaggtcgaccacgatcgccccacgctttctttccagagaacccaaaggaagacgttgttcgagtctccaccgatctcaacatcaatttctcggcgttcatgactgtcgttggaccagttccaccgtgtggcggctcgacaaatacgatgaatccacggggcagtacttcgtgaccatcggcggtgtcaaaggaaacccaggtcccgaaaccattagtagctggtttaagattgaggagttttgtggtagtggtttttacaagcttgttttctgtcccaccgtttgtggttcctgcaaagtaaaatgcggagatgtgggcatttacattgatcagaagggaagaaggcgtttggctctcagcgataaaccattcgcattcgagttcaacaaaaccgtatacttctaa

SEQ ID NO: 2 - protein, 220 aa, Artificial Sequence
  Synsepalum dulcificum
MKELTMLSLSFFFVSALLAAAANPLLSAADSAPNPVLDIDGEKLRTGTNYYIVPVLRDHGGGLTVSATTPNGTFVCPPRVVQTRKEVDHDRPLAFFPENPKEDVVRVSTDLNINFSAFMPCRWTSSTVWRLDKYDESTGQYFVTIGGVKGNPGPETISSWFKIEEFCGSGFYKLVFCPTVCGSCKVKCGDVGIYIDQKGRRRLALSDKPFAFEFNKTVYF

SEQ ID NO: 3 - DNA, 784 bp, Pisum sativum
tatgcaacttacaacgtgcactcgcggaggattggacgtgtgcaacttacaacgtacgcattgttcgttcatacaatagtgtagaattggagatgtgcaacttacaacatgtgcaacttacaacgtgcgctcgcggaggaatgtgaagttgaacacgtacaaacttacgtcatttgtgcatgcagaagcatagagctgagcacacaattcataatttgaaggacacatgatttgctataaagaactctttagaagtaccacaactttgactgagtttgatatagctaataaagatggagctcattataatttgaatggcataatgcaagctaaacgaacaagcttagttaatcatgttaaacaacaattctttgtaataataaaattgtctttcaactagtccaagtttatgagttgattcttcggaataaattagaaaatatcttagattttatacttcattgattatttcatagagcaagtaggagaaataaaaatatactagtattatttactaaaaaaaatctaagccacgtcggaggataacatccaacccagccaatcacagcaatgttcatcagataacccactttaagcccacgcactctgtggcacatctacattatctaaatcacatattcttccacacatcttagccacacaaaaacccaatccacatctttatcatccattctataaaatcaccttctgtgtgtctctctttcgattcccttcaaacacatacaaattcagtagagaagaaactcattactcttgagaaaaa

SEQ ID NO: 4 - DNA, 250 bp, Arabidopsis thaliana
atatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataag